(19) United States
(12) Patent Application Publication
(10) Pub. No.: US 2010/0081959 A1
(43) Pub. Date: Apr. 1, 2010

(54) DIAGNOSTIC APPARATUS

(76) Inventor: Vladimir Igorevic Nesterov, Moscow (RU)

Correspondence Address: VARNUM, RIDDERING, SCHMIDT & HOWLETT LLP, 333 BRIDGE STREET, NW, P.O. BOX 352, GRAND RAPIDS, MI, 49501-0352, US

(21) Appl. No.: 12/524,470

(22) PCT Filed: Jan. 25, 2008
(86) PCT No.: PCT/DE08/00133

§ 371 Date: Jul. 24, 2009

(30) Foreign Application Priority Data
Jan. 26, 2007 (DE) 10 2007 004 954.6

(51) Int. Cl. A61B 5/0484

(52) U.S. Cl. 600/544

(57) ABSTRACT

Diagnostic apparatus featuring biological feedback for diagnosing a patient (4), comprising: a data processing unit (1) which actuates a stimulus generating device (2, 3) so as to emit at least one predefined series of stimuli and by means of which the patient (4) can be exposed in a controlled manner to the at least one series of stimuli, and at least one measuring device (5, 6) which measures the reactions of the brainwaves of the patient (4) brought about by the stimuli applied to the patient (4), characterised by a unit (7) which is connected to the measuring device (5, 6) and to the stimulus generating device (2, 3) and which synchronises the reaction values and the series of stimuli, said unit being connected to the data processing unit (1) and forwarding the synchronised reaction values to the data processing unit (1) for evaluation purposes, and characterised in that the stimulus generating device (2, 3) has a carbon single crystal (3) which is arranged between electrodes and can be excited by electrical current pulses between the electrodes in order to indicate one of the series of stimuli to the patient (4).

The invention relates to a diagnostic apparatus featuring biological feedback according to the preamble of claim 1.

A diagnostic apparatus based on biological feedback is disclosed in U.S. Pat. No. 4,195,626. Therein, a patient is exposed to a series of different acoustic and visual stimuli and also electrical and tactile stimuli, and his reaction thereto is measured. During this, the patient is accommodated in a shielded chamber which is intended to reduce irritating stimuli from the environment. The series of stimuli are controlled by a microprocessor and subsequently corrected depending on the reaction of the patient. The disadvantage of the system is that the measured values are dependent on the state of consciousness of the patient.

U.S. Pat. No. 6,549,805 B1 discloses a diagnostic system in which an operator is integrated. Both the operator and the patient are exposed to the same generated series of stimuli. The reaction of the patient is evaluated in a central data processing unit. The central data processing unit comprises a stimulus generator for generating the series of stimuli for the patient and the operator. The initialisation device with a sensor detects the reaction of the brainwaves of the patient to the applied stimulus and transmits a digital signal to the data processing unit. Two biological feedback loops are provided in the system. On the one hand the stimulus generator, patient and initialisation device, and on the other hand the stimulus generator, operator and patient. In addition, the patient is exposed to optical pulses which are generated by a cadistor and which have the frequency of the theta wave of the patient's brain. The cadistor is placed just in front of the patient's forehead. It consists of a silicon semiconductor crystal and is excited by laser light of a certain wavelength in order to emit the stimuli which stimulate the brain of the patient. The invention is delimited with respect to this.

Significant disadvantages of the last-mentioned diagnostic apparatus are the fact that the reactions of the patient to the series of stimuli are small and that an operator, preferably a physician, is also required in addition to the patient in order to carry out the diagnosis. The apparatus is therefore not self-sufficient, and moreover diagnostic examinations from a distance and without medical staff are hardly possible. In addition, the sensitivity of the apparatus is not high.

The object of the invention is to provide a diagnostic apparatus featuring biological feedback which overcomes the abovementioned disadvantages.

This object is achieved by a diagnostic apparatus featuring biological feedback as mentioned in the introduction, having the characterising features of claim 1.

The diagnostic apparatus diagnoses the state of health of organs of the patient and also his overall state of health. A data processing unit having a processor actuates a stimulus generating device so as to emit at least one predefined series of stimuli, by means of which the patient can be exposed in a controlled manner to the at least one series of stimuli.

The stimulus generating device can generate different types of series of stimuli. It can generate optical stimuli, i.e. electromagnetic waves in the visible spectrum, or acoustic stimuli, but also magnetic stimuli and preferably electromagnetic stimuli at very low frequencies of up to 10 Hz.

A measuring device measures the intuitive reactions of the patient brought about by the stimuli applied to the patient. The patient's reaction is preferably measured based on the change in the brainwaves of the patient. Measurements of the intensity, change, etc. of brainwaves are known in principle.

According to the invention, the series of stimuli are firstly synchronised with the measured brain activities of the patient in a synchronisation unit. The synchronised stimuli and reaction measured values are forwarded to the data processing unit for evaluation purposes. By virtue of the synchronisation unit according to the invention, the reactions of the brainwaves can be better evaluated. Advantageously, there is no need for an operator, in particular medical staff, independent of the patient.

Preferably, the synchronisation unit is connected directly to the stimulus generator and also directly, i.e. without any interposition of further devices, to the measuring device.

In the data processing unit, preferably reaction values of the patient are separated from the stimulus values, and the reaction values are evaluated. Based on the reaction values, a diagnosis can be made and the state of health of the patient and also of individual organs can be determined.

For diagnosis, the patient is placed in a relaxed state and he is then exposed to the series of stimuli which act on the brainwaves of the patient directly, e.g. by electrical and/or magnetic fields acting directly on the brainwaves, or indirectly, e.g. by optical or acoustic stimuli acting on his brain via the sensory apparatus.

By means of the stimulus generating device, the patient is preferably stimulated with stimuli having a frequency in the range of the frequency of the theta waves of the human brain, preferably between 1 Hz and 9 Hz, most preferably between 3 Hz and 8 Hz. The measuring device measures the reaction of the brainwaves of the patient to the stimulus, by measuring the voltage curve of the brainwaves preferably in this frequency range. The reaction of the patient takes place subconsciously, intuitively. The patient need not consciously act for the diagnosis.

Healthy patients react to the series of stimuli in a comparable manner. In particular, shifts in the frequency of the stimuli on the one hand and of the brainwaves as a reaction to the stimulation on the other hand are then small. If the brain is stimulated for example at a frequency of 4.9 Hz, the measuring device measures as a reaction brainwaves of approximately also 4.9 Hz. On the other hand, frequency shifts indicate a pathological finding. The diagnostic apparatus according to the invention makes use of these determined relationships.

Explanations concerning this complicated process, which largely takes place in the patient's subconscious mind, are based on the fact that human organs can be assigned specific resonant frequencies due to their specific cell structure and associated molecular structure. Changes and disruptions to the tissue lead to a shift in the resonant frequencies.

Although it is conceivable to carry out the measurements on the organ directly, nevertheless it has been found that the measurements can be carried out better on the brain itself. There, the reactions of the patient are more intense and the measured values are higher. The brain or regions of the brain interact with the associated organs of the body. If the brain is excited at a resonant frequency assigned to an organ, in particular the associated organ is thus addressed and it generates a reaction which in the brain can in turn be measured in the brainwaves.

According to the invention, the stimulus generating device has a carbon single crystal which is arranged between electrodes and can be excited by electrical current pulses between the electrodes in order to indicate one of the series of stimuli to the patient. The carbon single crystal provided with electrodes is also referred to as the cadistor.

It has been found that the patient's reaction depends very sensitively on the type and generation of the stimuli. In an entirely surprising manner, it has been found that the excitation of a carbon single crystal by electrical pulses between two electrodes in the range of the frequency of the theta waves leads to much stronger reactions of the brainwaves of the patient than when the stimuli are generated by a silicon single crystal arranged between two electrodes. The reaction of the brainwaves of the patient is on average around 2.3 times greater than the reaction produced by the silicon cadistor.

In order to increase the accuracy of the measurements, a capacitance measuring instrument is provided as part of the measuring device, which monitors the biological reaction of the scalp to the stimuli. The reactions of the scalp are taken into account when evaluating the reaction measured values.

In one further development of the invention, the stimulus generating device comprises a set of headphones with two earcups, and the carbon single crystal is arranged in one of the earcups and the measuring device comprises a measuring sensor accommodated in the other earcup.

By arranging the cadistor in one earcup and arranging the measuring sensor in the other earcup, on the one hand both are as far away as possible from one another so as not to mutually influence one another directly, and on the other hand both devices are close enough to the patient's brain to excite his brainwaves on the one hand and to measure the reaction thereto on the other hand.

Advantageously, magnets which can be controlled in terms of their magnetic strength are arranged in the interior of the two earcups. The two magnets are oriented in pole opposition relative to the brain of the patient. Preferably, in one earcup, in which the cadistor is also arranged, the North pole is arranged on the side facing towards the patient, and in the other earcup, in which the measuring sensor is also accommodated, the South pole is arranged on the side facing towards the patient. Advantageously, the magnetic field strength of the two magnets is variable. The magnets can thus generate stimuli, preferably at a frequency in the range of the theta waves which corresponds to the frequency of the cadistor, so as to stimulate the brain.

The measuring sensor may comprise a trigger sensor which is capable of detecting even weak fluctuations that take place against a very noisy background.

Preferably, a telemetry module is provided which is connected to a data network, e.g. the Internet, and which allows the preferably encrypted transmission of the measured data to a remote PC, where a physician can make the diagnosis. The system is therefore advantageously locally independent of a physician.

The invention will be described on the basis of one example of embodiment in a FIGURE.

In the FIGURE:

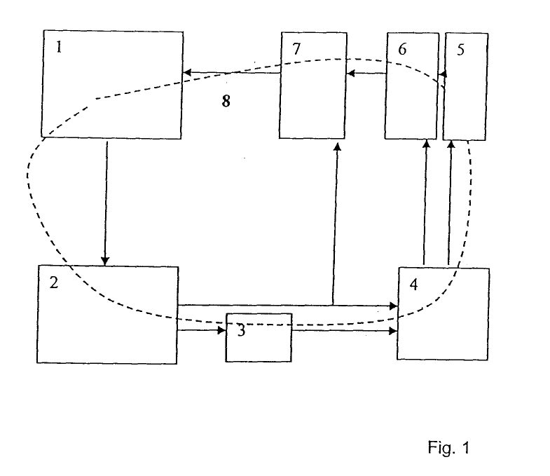

FIG. 1 shows a block diagram of the diagnostic apparatus according to the invention.

The diagnostic apparatus according to the invention comprises the data processing unit 1 having a microprocessor and a telemetry module, and a stimulus generator 2 which on the one hand excites a cadistor 3 to emit a first series of stimuli and on the other hand emits a further series of stimuli directly to the patient 4.

The measuring device 5, 6 which measures the change in the brainwaves of the patient 4 comprises a capacitive measuring instrument 5 for determining disturbing side effects through the reaction of the scalp of the patient 4 to the stimuli, and the actual measuring sensor 6. The capacitive measuring instrument 5 monitors and measures the biological reaction of the scalp of the patient 4. The reactions of the scalp of the patient 4 are taken into account when evaluating the reaction measured values.

In this example of embodiment, the cadistor 3 is arranged in the left earcup (not shown) of a set of headphones (not shown) and the measuring sensor 6 is provided in the right earcup of the set of headphones. The patient 4 positions the set of headphones in the conventional manner during the diagnosis. The cadistor 3 is then located in the region of his left ear and the measuring sensor at his right ear.

The cadistor 3 serves to amplify the intuitive reaction of the patient. The cadistor 3 consists of two electrodes, between which a carbon single crystal (simulated diamond) is arranged. The stimulus generator 2 applies current pulses to the two electrodes. The frequency at which the excitation of the carbon single crystal takes place is varied from approximately 1 Hz to approximately 8 Hz. By virtue of the current pulses, the carbon single crystal is excited and emits in particular electromagnetic radiation in pulse form. The frequency of the pulses is also in the range between 1 Hz and 8 Hz, which is the range of the frequency of the theta waves of the brain of the patient 4.

The cadistor 3 is excited by the stimulus generator 2 and emits a series of stimuli which stimulates the brain of the patient 4. The intuitive reaction of the brain in the form of changes in the brainwaves of the patient 4 can be measured by the measuring sensor 6.

The design of the cadistor 3 as a carbon single crystal makes it possible to amplify the intuitive reaction of the patient 4 by on average 2.3 times compared to the silicon-based cadistor version described in U.S. Pat. No. 6,549,805 B1. Overall, an increase in the accuracy and sensitivity of the diagnosis is possible compared to the aforementioned prior art.

The measuring sensor 6 measures the reaction signal of the brainwaves of the patient 4 to the stimulus applied thereto. It is provided in the right earcup of the set of headphones. It picks up this biological reaction of the patient 4 as an analogue signal.

Furthermore, a magnet which can be controlled in terms of its magnetic strength is provided in each of the two earcups of the set of headphones. The magnetic strength of the two magnets is controlled by the stimulus generator 2. In the left earcup the magnet is arranged with its North pole directly at the head of the patient 4, and in the right earcup the magnet is arranged with its South pole directly at the head of the patient 4. The magnetic strength of the magnets is controlled by the stimulus generator 2. The magnets thus generate stimuli, controlled by the stimulus generator 2, on the brain of the patient 4.

The signal picked up by the measuring sensor 6 is amplified. Preferably a differential amplification takes place, the amplification coefficient of which is −60 dB.

In order to isolate the intuitive reaction of the patient 4 to the stimuli through the magnets and the cadistor 3, use is made of a synchronisation unit 7 which synchronises the stimuli brought about by the stimulus generator 2 and the reaction values measured by the measuring sensor 6, and thus makes any differences between the stimuli and the reactions more easily detectable in the data processing unit 1. For this, the stimulus signal and the picked-up signal are compared. The final processing of the picked-up signal and also the evaluation take place in the data processing unit 1.

Signals produced by the biological reaction of the scalp as a result of activation of brain activity by the stimuli are superposed on the signal picked up by the measuring sensor 6. In order to monitor and take account of these signals, the capacitive measuring instrument 5 is provided in addition to the measuring sensor 6. The measuring sensor 6 is a trigger sensor which is supplied with a variable current between 1.0 μA and 1.5 μA. The variation of the current takes place in a manner proportional to the change in the capacitive component of the scalp of the patient 4.

The data processing unit 1 makes it possible to ascertain the bioelectrical activity of the brain neurons by filtering out weak signals against the background of statistical fluctuations and carrying out amplification. Faintly noticeable changes in the signals are obtained from mean statistical characteristic noise values of the measured fields and are digitised by the data processing unit 1 and optionally forwarded via a telemetry module to a communication system.

The reaction values evaluated in the data processing unit 1 are re-used to actuate the stimulus generator 2. It forms a circuit 8 with feedback along the data processing unit 1, stimulus generator 2, cadistor 3, patient 4, measuring device 5, 6 with measuring sensor 6 and capacitive measuring instrument 5, and synchronisation unit 7.

The telemetry module comprises a video camera, a microphone, a video link and preferably a decoding unit, and also an acoustic system and equipment for video playback. These are installed in the apparatus.

The apparatus offers the possibility of audiovisual contact between the patient 4 and the physician while carrying out the diagnosis, even if the physician is located far from the patient 4.

The apparatus also offers the possibility of obtaining access to the diagnostic program while carrying out the diagnosis via a computer. In this case, the evaluation of the data may take place on a remote computer. The torsion diagnosis system is equipped with programs which generate graphic files directly from the measured values. The data material obtained can be sent via e-mail or a special server to the treating medical centre. The transmission of the e-mail data may take place in an encrypted manner. For medical consultation and transmission of the data, the communication program package TorDi was developed which, in addition to programs for carrying out the diagnosis itself, also contains e-mail systems and calendar and scheduling system programs for automating business procedures. TorDi contains an e-mail database for storing documents. It has the function of a web server. TorDi has properties of a document management system with integrated communication possibilities. TorDi runs on the Windows operating system, supporting the TCP/EP protocol and data transmission via analogue telephone lines. Access to the server can take place via a standard web browser. TorDi allows use of the Internet as a means for patient/server communication. The data in the TorDi database can be converted into an HTML page. As access to the server, use can be made of global and also local networks and remote access via a telephone line with modem.

### LIST OF REFERENCES

1 data processing unit (microprocessor and telemetry module)
2 stimulus generator
3 cadistor/carbon single crystal
4 patient
5 capacitive measuring instrument
6 measuring sensor
7 synchronisation unit
8 circuit

1. Diagnostic apparatus featuring biological feedback for diagnosing a patient (4), comprising:
a data processing unit (1) which actuates a stimulus generating device (2, 3) so as to emit at least one predefined series of stimuli and by means of which the patient (4) can be exposed in a controlled manner to the at least one series of stimuli, and
at least one measuring device (5, 6) which measures the reactions of the brainwaves of the patient (4) brought about by the stimuli applied to the patient (4), characterised by a unit (7) which is connected to the measuring device (5, 6) and to the stimulus generating device (2, 3) and which synchronises the reaction values and the series of stimuli, said unit being connected to the data processing unit (1) and forwarding the synchronised reaction values to the data processing unit (1) for evaluation purposes, and characterised in that the stimulus generating device (2, 3) has a carbon single crystal (3) which is arranged between electrodes and can be excited by electrical current pulses between the electrodes in order to indicate one of the series of stimuli to the patient (4).

2. Diagnostic apparatus according to claim 1, characterised in that the stimulus generating device (2, 3) comprises a set of headphones with two earcups, and the carbon single crystal (3) is arranged in one of the earcups and the measuring device (5, 6) comprises a measuring sensor (6) accommodated in the other earcup.

3. Diagnostic apparatus according to claim 2, characterised in that magnets which can be controlled in terms of their magnetic strength are arranged in the interior of the two earcups.

4. Diagnostic apparatus according to claim 3, characterised in that in one earcup the North pole is arranged on the side facing towards the patient and in the other earcup the South pole is arranged on the side facing towards the patient.

5. Diagnostic apparatus according to claim 1, characterised in that the synchronisation unit (7) is connected directly both to the stimulus generator (2) and also directly to the measuring device (5, 6).

6. Diagnostic apparatus according to claim 1, characterised in that the series of stimuli emitted by the carbon single crystal (3) are pulses of electromagnetic waves which, in terms of their pulse frequency, essentially correspond to the theta rhythm of the brainwaves of the patient (4).

7. Diagnostic apparatus according to claim 2, characterised in that the measuring sensor (6) comprises a trigger sensor.

8. Diagnostic apparatus according to claim 1, characterised by a capacitance measuring instrument (5) for the scalp of the patient (4), said capacitance measuring instrument being connected to the measuring device (5, 6).

9. Diagnostic apparatus according to claim 1, characterised in that the data processing unit (1) comprises a telemetry module with a connection for an information network for transmitting data.
